SEQUENCE LISTING (14 sequences)

SEQ ID NO: 1 - protein, 6 aa, Artificial Sequence
  Synthetic Polypeptide
PLGLAG

SEQ ID NO: 2 - protein, 7 aa, Artificial Sequence
  Synthetic Polypeptide
PQGIAGW

SEQ ID NO: 3 - protein, 7 aa, Artificial Sequence
  Synthetic Polypeptide
KPLGLAR

SEQ ID NO: 4 - protein, 7 aa, Artificial Sequence
  Synthetic Polypeptide
PLGMYSR

SEQ ID NO: 5 - protein, 6 aa, Artificial Sequence
  Synthetic Polypeptide
PLGMSR

SEQ ID NO: 6 - protein, 8 aa, Artificial Sequence
  Synthetic Polypeptide
  MISC_FEATURE (1)..(1): Mca
  MISC_FEATURE (8)..(8): (Dap{Dnp)(K{N3)-CONH2
KGPLGLAG

SEQ ID NO: 7 - protein, 7 aa, Artificial Sequence
  Synthetic Polypeptide
  MISC_FEATURE (1)..(1): Mca
  MISC_FEATURE (7)..(7): (Dap{Dnp)G(K{N3)-CONH2
KGPLGLA

SEQ ID NO: 8 - protein, 7 aa, Artificial Sequence
  Synthetic Polypeptide
  MISC_FEATURE (1)..(1): Abz
  MISC_FEATURE (7)..(7): (Y{NO2)C-COOH
KPLGLAG

SEQ ID NO: 9 - protein, 9 aa, Artificial Sequence
  Synthetic Polypeptide
  MISC_FEATURE (1)..(1): Ch3
  MISC_FEATURE (9)..(9): (K{N3)-CONH2
KGPLGLAGG

SEQ ID NO: 10 - protein, 10 aa, Artificial Sequence
  Synthetic Polypeptide
  MISC_FEATURE (1)..(1): CH3
  MISC_FEATURE (10)..(10): CONH2
KGPLGLAGGC

SEQ ID NO: 11 - DNA, 20 bp, Artificial Sequence
  Synthetic Polynucleotide
  misc_feature (1)..(1): C8 Alkyne
  misc_feature (20)..(20): Sp18-Sp18-SH
tttttttttttttttttttt

SEQ ID NO: 12 - DNA, 20 bp, Artificial Sequence
  Synthetic Polynucleotide
  misc_feature (1)..(1): Hexynyl
  misc_feature (20)..(20): Sp18-Sp18-SH
tttttttttttttttttttt

SEQ ID NO: 13 - protein, 20 aa, Artificial Sequence
  Synthetic Polynucleotide
  MISC_FEATURE (1)..(1): C8 Alkyne-Cy5
  MISC_FEATURE (20)..(20): Sp18-Sp18-SH
TTTTTTTTTTTTTTTTTTTT

SEQ ID NO: 14 - DNA, 20 bp, Artificial Sequence
  Synthetic Polynucleotide
  misc_feature (1)..(1): Cy3.5
  misc_feature (20)..(20): Sp18-Sp18-SH
tttttttttttttttttttt

(19) United States
(12) Patent Application Publication
(10) Pub. No.: US 2020/0246484 A1
(43) Pub. Date: Aug. 6, 2020

(54) SPHERICAL NUCLEIC ACIDS (SNAS) WITH SHEDDABLE PEG LAYERS

(71) Applicant: NORTHWESTERN UNIVERSITY, Evanston, IL (US)

(72) Inventors: Chad A. Mirkin, Wilmette, IL (US); Brian R. Meckes, Highland Village, TX (US); Wuliang Zhang, Evanston, IL (US)

(21) Appl. No.: 16/753,269

(22) PCT Filed: Oct. 3, 2018
(86) PCT No.: PCT/US18/54221

§ 371 Date: Apr. 2, 2020

Related U.S. Application Data
(60) Provisional application No. 62/567,603, filed on Oct. 3, 2017.

(51) Int. Cl. A61K 47/69; C07K 7/06; A61K 47/10

(52) U.S. Cl. CPC A61K 47/6929; A61K 47/10; C07K 7/06; A61K 47/6923

(57) ABSTRACT

The present disclosure provides compositions and methods directed to the synthesis and use of SNAs with a sheddable PEG layer that incorporates an enzyme-sensitive linker.

### CROSS REFERENCE TO RELATED APPLICATIONS

This application claims the priority benefit under 35 U.S.C. § 119(e) of U.S. Provisional Patent Application No. 62/567,603, filed Oct. 3, 2017, the disclosure of which is incorporated herein by reference in its entirety.

### STATEMENT OF GOVERNMENT INTEREST

This invention was made with government support under U54 CA199091 awarded by the National Institutes of Health. The government has certain rights in the invention.

### INCORPORATION BY REFERENCE OF MATERIALS SUBMITTED ELECTRONICALLY

This application contains, as a separate part of the disclosure, a Sequence Listing in computer readable form (Filename: 2017-169_Seqlisting.txt; Size: 4,166 bytes; Created: Oct. 3, 2018), which is incorporated by reference in its entirety.

### SUMMARY

Spherical nucleic acids (SNAs), a nanomaterial consisting of a spherical core densely functionalized with highly oriented nucleic acids, have enhanced properties compared to their linear counterparts. These properties include increased resistance to nuclease degradation, ready cellular uptake, and increased binding affinity to complementary strands, which makes SNAs appealing for many biological and therapeutic applications. However, SNAs have short blood circulation half-lives that limit their systemic delivery. Coating these structures with chemically inert molecules, such as polyethylene glycol (PEG), can enhance the circulation life of these molecules, but reduces the uptake efficiency.

Accordingly, the present disclosure is directed to the synthesis and use of SNAs with a sheddable PEG layer that incorporates an enzyme-sensitive peptide linker. Such an enzyme is, in some embodiments, overexpressed in a tumor microenvironment. This structure is designed to have a PEG coating while circulating to decrease clearance of the nanoparticles. Upon entering a tumor environment, the PEG coating is actively shed by cleavage of the peptide substrates by a tumor-associated enzyme, thus allowing the nanoparticles to take advantage of the properties of the SNA. Further, the FDA has approved the use of PEG modifications in a number of different therapeutic formulations.

Applications of the technology disclosed herein include providing formulations of nanotherapies for treatment of solid tumors, creating active components for altering the biological behavior of SNAs, and extending the circulation time of SNA therapies. An advantage provided by the disclosure is the provision of a SNA that enjoys the benefits of a PEG coating while being converted into an active SNA once in a tumor microenvironment. Another advantage provided by the disclosure is the utilization of biocompatible and specific peptide sequences that allow tailoring to subsets of tumor associated enzymes. A further advantage is that the SNAs of the disclosure provide targeted delivery of therapeutics based on local protein expression.

Accordingly, in some aspects the disclosure provides a nanoparticle having an oligonucleotide functionalized thereto, the oligonucleotide comprising polyethylene glycol (PEG) and/or a peptide, configured as follows:

nanoparticle------oligonucleotide------peptide------PEG.

In some embodiments, the nanoparticle comprises an oligonucleotide comprising a peptide, wherein the oligonucleotide does not include PEG. In some of these embodiments, it is contemplated that the oligonucleotide additionally comprises a targeting ligand. In further embodiments, the targeting ligand is an antibody, a mimetic peptide, or a protein.

In some embodiments, the nanoparticle further comprises a conjugate functionalized to the nanoparticle, wherein the conjugate comprises a spacer, a peptide and PEG, configured as follows:

nanoparticle------spacer------peptide------PEG.

In further embodiments, the spacer comprises PEG or an amino acid. In some embodiments, the spacer is shorter in length than the oligonucleotide. In further embodiments, the peptide is enzyme-sensitive. In some embodiments, the enzyme is present in a tumor microenvironment (TME). In further embodiments, the enzyme is a matrix metallo-proteinase (MMP). In still further embodiments, the MMP is MMP-2 and/or MMP-9. In some embodiments, the peptide sequence is PLGLAG (SEQ ID NO: 1), PQGIAGW (SEQ ID NO: 2), KPLGLAR (SEQ ID NO: 3), PLGMYSR (SEQ ID NO: 4), or PLGMSR (SEQ ID NO: 5).

In various embodiments, the nanoparticle is organic or inorganic. In some embodiments, the nanoparticle is metallic. In further embodiments, the nanoparticle comprises gold, silver, platinum, aluminum, palladium, copper, cobalt, indium, or nickel. In some embodiments, the nanoparticle is a liposome.

In some embodiments, the nanoparticle further comprises an agent.

In some aspects, the disclosure provides a composition comprising a nanoparticle of the disclosure and a pharmaceutically acceptable carrier. In some embodiments, the composition further comprises an agent.

In some aspects, the disclosure provides a method of modulating gene expression comprising administering to a cell a nanoparticle of the disclosure.

Additional aspects and embodiments of the disclosure are described in the following enumerated paragraphs.

Paragraph 1. A nanoparticle having an oligonucleotide functionalized thereto, the oligonucleotide optionally comprising polyethylene glycol (PEG) and/or a peptide, configured as follows:

nanoparticle------oligonucleotide------peptide------PEG.

Paragraph 2. The nanoparticle of paragraph 1, further comprising a conjugate functionalized to the nanoparticle, wherein the conjugate comprises a spacer, a peptide and PEG, configured as follows:

nanoparticle------spacer------peptide------PEG.

Paragraph 3. The nanoparticle of paragraph 2, wherein the spacer comprises PEG or an amino acid.

Paragraph 4. The nanoparticle of paragraph 3, wherein the spacer is shorter in length than the oligonucleotide.

Paragraph 5. The nanoparticle of any one of paragraphs 1-4, wherein the peptide is enzyme-sensitive.

Paragraph 6. The nanoparticle of paragraph 5, wherein the enzyme is present in a tumor microenvironment (TME).

Paragraph 7. The nanoparticle of paragraph 5 or paragraph 6, wherein the enzyme is a matrix metallo-proteinase (MMP).

Paragraph 8. The nanoparticle of paragraph 7, wherein the MMP is MMP-2 and/or MMP-9.

Paragraph 9. The nanoparticle of paragraph 8, wherein the peptide sequence is PLGLAG (SEQ ID NO: 1), PQGIAGW (SEQ ID NO: 2), KPLGLAR (SEQ ID NO: 3), PLGMYSR (SEQ ID NO: 4), or PLGMSR (SEQ ID NO: 5).

Paragraph 10. The nanoparticle of any one of paragraphs 1-9, wherein the nanoparticle is organic or inorganic.

Paragraph 11. The nanoparticle of paragraph 10, wherein the nanoparticle is metallic.

Paragraph 12. The nanoparticle of paragraph 11, wherein the nanoparticle comprises gold, silver, platinum, aluminum, palladium, copper, cobalt, indium, or nickel.

Paragraph 13. The nanoparticle of paragraph 10, wherein the nanoparticle is a liposome.

Paragraph 14. A method of modulating gene expression comprising administering to a cell the nanoparticle of any one of paragraphs 1-13.

### BRIEF DESCRIPTION OF THE FIGURES

FIG. 1 shows a scheme of PEG functionalized-SNA. An SNA is functionalized with a dense and highly oriented shell of nucleic acids. PEG is covalently attached to the SNA with a peptide linker that is recognized and cleaved by MMPs. When the SNA enters the tumor microenvironment (TME), the overexpressed MMPs cleave the PEG layer, revealing an SNA that can be taken up by cells. The SNA taken up by the cell can then perform gene regulation.

FIG. 2 depicts a schematic of the two assembly approaches for creating enzyme cleavable PEG shells on SNAs. Strategy 1 shows the direct attachment to oligonucleotides while strategy 2 shows backfilling around the oligonucleotides.

FIG. 3 depicts a synthesis scheme for attaching peptide to NSH-functionalized PEG.

FIG. 4 shows a functionalization scheme for conjugating peptide-PEG to oligonucleotides.

FIG. 5 shows MALDI-TOF Mass spectrum of DNA A) before and B) after conjugation to the MMP cleavable peptide.

FIG. 6 depicts a scheme of the fluorophore quenching design in the peptide sequence.

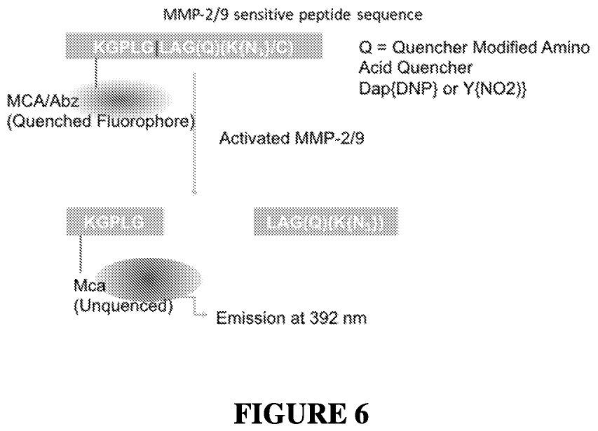

FIG. 7 shows the quantification of ligand loading density of oligonucleotides (black) and conjugates (grey) on Au nanoparticles. SNAs were formulated with DNA-peptide-PEG2K conjugates. SNA=spherical nucleic acid: L-2K=low density cleavable PEGylated SNA: D-2K=low density noncleavable PEGylated SNA: L-5K=high density cleavable PEGylated SNA: D-5K=high density noncleavable PEGylated SNA.

FIG. 8 shows the quantification of ligand loading density of oligonucleotides (black) and conjugates (grey) on Au nanoparticles. SNAs were formulated with peptide PEG2K, PEG5K, PEG10K conjugates attached directly to the Au core.

FIG. 9 shows MMP9-mediated cleavage kinetics of the cleavable (1′)/non-cleavable (‘D’) peptides attached to PEG2K, PEG5K, and PEG10K and functionalized onto SNAs.

FIG. 10 displays the uptake of peptide-PEG2K, peptide-PEG5K and peptide-PEG10K functionalized SNAs into U87 cells after 30 minute and 4 hour incubation, without MMP pretreatment. Notably, these structures have lower PEG densities (approximately 50 PEG/AuNP and approximately 125 oligonucleotides/AuNP; FIG. 9) than the structures used in FIG. 9.

FIG. 11 depicts U87 cell uptake of high-density peptide-PEG2K (90 PEGs/AuNP) functionalized SNAs after four-hour incubation, with and without MMP-9 pretreatment.

FIG. 12 shows the Au content in blood 1, 6, and 24 hours post systemic administration of SNAs as well as cleavable and non-cleavable PEG2K functionalized SNAs into glioma-bearing mice.

FIG. 13 shows SNA accumulation in select organs.

### DESCRIPTION

The present disclosure provides spherical nucleic acids (SNAs), nanostructures comprising either an inorganic (e.g., metallic (see, e.g., U.S. Patent Application Publication No. 2009/0209629, incorporated herein by reference in its entirety)), a hollow nanoparticle as disclosed in U.S. Patent Application Publication No. 2012/0282186 (incorporated herein by reference in its entirety), or an organic spherical core (e.g., lipids (see, e.g., U.S. Patent Application Publication No. 2016/0310425, incorporated herein by reference in its entirety)) functionalized with a dense and highly oriented nucleic acids shells, are synthesized with a polyethylene glycol (PEG) shell that is functionalized to the nanoparticle using an enzyme cleavable peptide linker.

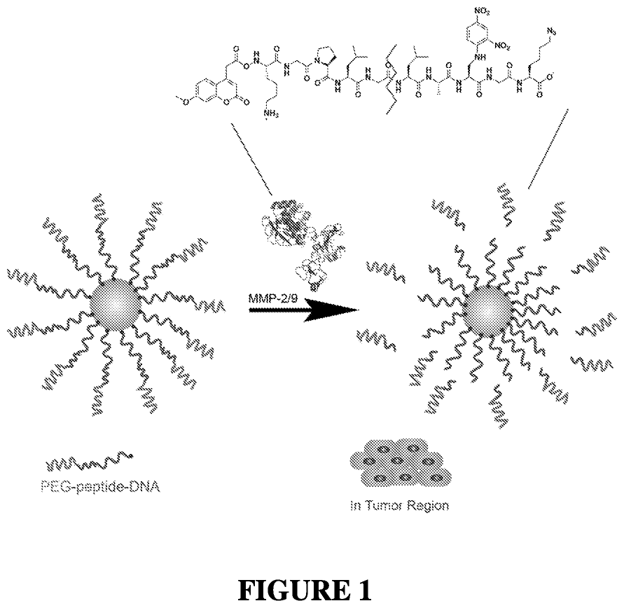

The nucleic acids utilized in the synthesis of the SNA include specific sequences that can be used to regulate the expression of specific proteins by cells to modulate cell behavior (e.g., slow proliferation, induce cell death). The use of a PEG layer reduces the non-specific adhesion of proteins that can degrade the nucleic acids, interferes with cell recognition, or increases clearance, which can lead to increased systemic circulation time and more stable SNAs. However, the PEG shell can also reduce cell uptake, potentially reducing the efficacy of SNAs. In order to regain functionality of SNAs, such as high cellular uptake, the cleavable linker attaching PEG to the SNA consists of a peptide sequence that is recognized and cleaved by specific enzymes, (which, in some embodiments, is a matrix metallo-proteinase (MMP)), that are overexpressed within tumor microenvironments (TMEs). The use of an enzyme-cleavable linker allows the PEG layer to be shed by the SNA upon entering the TME, facilitating the uptake of SNAs by cells. See FIG. 1.

Spherical Nucleic Acids.

Spherical Nucleic Acids (SNAs) are nanostructures consisting of a spherical nanoparticle core functionalized with a dense and highly oriented nucleic acid shell [Cutler et al., Journal of the American Chemical Society 2012, 134 (3), 1376-1391; Mirkin et al., Nature 1996, 382 (6592), 607-609; Banga et al., J. Am. Chem. Soc. 2014, 136 (28), 9866-9869; Banga et al., J. Am. Chem. Soc. 2017; Zheng et al., ACS Nano 2013, 7 (8), 6545-6554; Brodin et al., J. Am. Chem. Soc. 2015, 137 (47), 14838-14841; Calabrese et al., Angew. Chem., Int. Ed. Engl. 2015, 54 (2), 476-480]. The dense and highly oriented nucleic acid shell allows SNAs to readily enter cells without transfecting agents [Rosi et al., Science 2006, 312 (5776), 1027-1030], increases the oligonucleotide affinity for complementary strands [Mirkin et al., Nature 1996, 382 (6592), 607-609], and decreases susceptibility to nucleases compared to their linear counterparts [Rosi et al., Science 2006, 312 (5776), 1027-1030]. Since the enhanced properties of the nucleic acids arises from their arrangement and density and not the type of template, SNAs can be synthesized using a variety of organic and inorganic spherical templates that include gold [Mirkin et al., Nature 1996, 382 (6592), 607-609], silver [Lee et al., Nano Lett. 2007, 7 (7), 2112-2115], infinite coordination polymers [Calabrese et al., Angew. Chem., Int. Ed. Engl. 2015, 54 (2), 476-480], proteins [Brodin et al., J. Am. Chem. Soc. 2015, 137 (47), 14838-14841], and lipids [Banga et al., J. Am. Chem. Soc. 2014, 136 (28), 9866-9869; Banga et al., J. Am. Chem. Soc. 2017]. For many biological and potential therapeutic applications, gold and lipidic structures have been pursued [Sprangers et al., Small 2016, 13; Radovic-Moreno et al., Proc. Natl. Acad. Sci. U.S.A 2015, 112 (13), 3892-3897; Jensen et al., Sci. Transl. Med. 2013, 5 (209), 209ra152-209ra152; Giljohann et al., J. Am. Chem. Soc. 2009, 131 (6), 2072-2073; Seferos et al., J. Am. Chem. Soc. 2007, 129 (50), 15477-15479; Zheng et al., Nano Lett. 2009, 9 (9), 3258-3261]. Previous studies have shown that density of the shell is critical to cellular interactions as higher oligonucleotide densities leads to increased uptake [Giljohann et al., Nano Letters 2007, 7 (12), 3818-3821]. Despite the rapid uptake of SNAs by cells, biodistribution remains a significant challenge. In vivo studies show that SNAs have a limited blood circulation half-life, less than one minute [Jensen et al., Sci. Transl. Med. 2013, 5 (209), 209ra152-209ra152]. A common strategy to overcome these shortcomings is through functionalization of the particles with polyethylene glycol (PEG) to prevent interactions of with serum proteins [Manson et al., Gold Bulletin 2011, 44 (2), 99-105; Veiseh et al., Nano Lett. 2005, 5 (6), 1003-1008; Fang et al., Small 2009, 5 (14), 1637-1641; Otsuka et al., Adv. Drug Delivery. Rev. 2012, 64, 246-255]. SNAs have been synthesized with PEG backfills and it was discovered that higher loading of PEG increased the circulation half-life, but decreased cellular uptake in vitro [Chinen et al., Bioconjugate Chemistry 2016, 27 (11), 2715-2721]. In order to overcome the limitations of SNAs and PEG, the SNAs provided herein are synthesized by attaching PEG to the nanoparticle surface utilizing tumor-associated protease cleavable linkers. Importantly, the properties of these SNAs (e.g., protein absorption, circulation time, PEG removal kinetics) can be modulated by tuning the peptide cleavage sequence (for example and without limitation, peptide cleavage sequences contemplated by the disclosure include PLGLAG (SEQ ID NO: 1), PQGIAGW (SEQ ID NO: 2), KPLGLAR (SEQ ID NO: 3), PLGMYSR (SEQ ID NO: 4), and PLGMSR (SEQ ID NO: 5), altering the PEG molecular weight/length (for example and without limitation, from about 200 Daltons to about 10,000 Daltons), and changing the PEG density (for example and without limitation, from about 1.5 to about 63 pmol/cm²). These SNAs demonstrated increased half-life in blood and better accumulation in tumors, while maintaining high cellular uptake into cancer cells, a characteristic intrinsic to SNAs.

Spherical nucleic acids (SNAs) comprise densely functionalized and highly oriented polynucleotides on the surface of a nanoparticle that can either be organic (e.g., a liposome) inorganic (e.g., gold, silver, or platinum) or hollow (e.g., silica-based). The spherical architecture of the polynucleotide shell confers unique advantages over traditional nucleic acid delivery methods, including entry into nearly all cells independent of transfection agents and resistance to nuclease degradation. Furthermore, SNAs can penetrate biological barriers, including the blood-brain (see, e.g., U.S. Patent Application Publication No. 2015/0031745, incorporated by reference herein in its entirety) and blood-tumor barriers as well as the epidermis (see, e.g., U.S. Patent Application Publication No. 2010/0233270, incorporated by reference herein in its entirety).

Nanoparticles are therefore provided which are functionalized to have a polynucleotide attached thereto. In general, nanoparticles contemplated include any compound or substance with a high loading capacity for a polynucleotide as described herein, including for example and without limitation, a metal, a semiconductor, a liposomal particle, insulator particle compositions, and a dendrimer (organic versus inorganic).

Thus, nanoparticles are contemplated which comprise a variety of inorganic materials including, but not limited to, metals, semi-conductor materials or ceramics as described in U.S. Patent Publication No 20030147966. For example, metal-based nanoparticles include those described herein. Ceramic nanoparticle materials include, but are not limited to, brushite, tricalcium phosphate, alumina, silica, and zirconia. Organic materials from which nanoparticles are produced include carbon. Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g., polypeptides such as BSA, polysaccharides, etc.), other biological materials (e.g., carbohydrates), and/or polymeric compounds are also contemplated for use in producing nanoparticles.

Liposomal particles, for example as disclosed in International Patent Application No. PCT/US2014/068429 (incorporated by reference herein in its entirety, particularly with respect to the discussion of liposomal particles) are also contemplated by the disclosure. Hollow particles, for example as described in U.S. Patent Publication Number 2012/0282186 (incorporated by reference herein in its entirety) are also contemplated herein. Liposomal particles of the disclosure have at least a substantially spherical geometry, an internal side and an external side, and comprise a lipid bilayer. The lipid bilayer comprises, in various embodiments, a lipid from the phosphocholine family of lipids or the phosphoethanolamine family of lipids. While not meant to be limiting, the first-lipid is chosen from group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), cardiolipin, lipid A, and a combination thereof.

In some aspects, the nanoparticle is metallic, and in various embodiments, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the invention include, also without limitation, ZnS, ZnO, Ti, TiO2, Sn, SnO2, Si, SiO₂, Fe, Fe+4, Ag, Cu, Ni, Al, steel, cobalt-chrome alloys, Cd, titanium alloys, AgI, AgBr, HgI2, PbS, PbSe, ZnTe, CdTe, In2S3, In2Se3, Cd3P2, Cd3As2, InAs, and GaAs. Methods of making ZnS, ZnO, TiO2, AgI, AgBr, HgI2, PbS, PbSe, ZnTe, CdTe, In2S3, In2Se3, Cd3P2, Cd3As2, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky, et al., J. Am. Chem. Soc., 112, 9438 (1990); Ushida et al., J. Phys. Chem., 95, 5382 (1992).

In practice, methods of increasing cellular uptake and inhibiting gene expression are provided using any suitable particle having oligonucleotides attached thereto that do not interfere with complex formation, i.e., hybridization to a target polynucleotide. The size, shape and chemical composition of the particles contribute to the properties of the resulting oligonucleotide-functionalized nanoparticle. These properties include for example, optical properties, optoelectronic properties, electrochemical properties, electronic properties, stability in various solutions, magnetic properties, and pore and channel size variation. The use of mixtures of particles having different sizes, shapes and/or chemical compositions, as well as the use of nanoparticles having uniform sizes, shapes and chemical composition, is contemplated. Examples of suitable particles include, without limitation, nanoparticles particles, aggregate particles, isotropic (such as spherical particles) and anisotropic particles (such as non-spherical rods, tetrahedral, prisms) and core-shell particles such as the ones described in U.S. patent application Ser. No. 10/034,451, filed Dec. 28, 2002, and International Application No. PCT/US01/50825, filed Dec. 28, 2002, the disclosures of which are incorporated by reference in their entirety.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers)

Suitable nanoparticles are also commercially available from, for example, Ted Pella, Inc. (gold), Amersham Corporation (gold) and Nanoprobes, Inc. (gold).

Also as described in US Patent Publication No. 20030147966, nanoparticles comprising materials described herein are available commercially or they can be produced from progressive nucleation in solution (e.g., by colloid reaction), or by various physical and chemical vapor deposition processes, such as sputter deposition. See, e.g., HaVashi, (1987) Vac. Sci. Technol. July/August 1987, A5(4):1375-84; Hayashi, (1987) Physics Today, December 1987, pp. 44-60; MRS Bulletin, January 1990, pgs. 16-47.

As further described in U.S. Patent Publication No. 20030147966, nanoparticles contemplated are produced using HAuCl₄ and a citrate-reducing agent, using methods known in the art. See, e.g., Marinakos et al., (1999) Adv. Mater. 11: 34-37; Marinakos et al., (1998) Chem. Mater. 10: 1214-19; Enustun & Turkevich, (1963) J. Am. Chem. Soc. 85: 3317. Tin oxide nanoparticles having a dispersed aggregate particle size of about 140 nm are available commercially from Vacuum Metallurgical Co., Ltd. of Chiba, Japan. Other commercially available nanoparticles of various compositions and size ranges are available, for example, from Vector Laboratories, Inc. of Burlingame, Calif.

Nanoparticles contemplated by the disclosure can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or the amount of surface area that can be functionalized as described herein. In further embodiments, a plurality of SNAs (e.g., liposomal particles) is produced and the SNAs in the plurality have a mean diameter of less than or equal to about 50 nanometers (e.g., about 5 nanometers to about 50 nanometers, or about 5 nanometers to about 40 nanometers, or about 5 nanometers to about 30 nanometers, or about 5 nanometers to about 20 nanometers, or about 10 nanometers to about 50 nanometers, or about 10 nanometers to about 40 nanometers, or about 10 nanometers to about 30 nanometers, or about 10 nanometers to about 20 nanometers). In further embodiments, the SNAs in the plurality created by a method of the disclosure have a mean diameter of less than or equal to about 20 nanometers, or less than or equal to about 25 nanometers, or less than or equal to about 30 nanometers, or less than or equal to about 35 nanometers, or less than or equal to about 40 nanometers, or less than or equal to about 45 nanometers.

Polynucleotides.

The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Nanoparticles provided that are functionalized with a polynucleotide, or a modified form thereof generally comprise a polynucleotide from about 5 nucleotides to about 100 nucleotides in length. More specifically, nanoparticles are functionalized with a polynucleotide that is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all polynucleotides intermediate in length of the sizes specifically disclosed to the extent that the polynucleotide is able to achieve the desired result. Accordingly, polynucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 125, about 150, about 175, about 200, about 250, about 300, about 350, about 400, about 450, about 500 or more nucleotides in length are contemplated.

In some embodiments, the polynucleotide attached to a nanoparticle is DNA. When DNA is attached to the nanoparticle, the DNA is in some embodiments comprised of a sequence that is sufficiently complementary to a target region of a polynucleotide such that hybridization of the DNA polynucleotide attached to a nanoparticle and the target polynucleotide takes place, thereby associating the target polynucleotide to the nanoparticle. The DNA in various aspects is single stranded or double-stranded, as long as the double-stranded molecule also includes a single strand region that hybridizes to a single strand region of the target polynucleotide. In some aspects, hybridization of the polynucleotide functionalized on the nanoparticle can form a triplex structure with a double-stranded target polynucleotide. In another aspect, a triplex structure can be formed by hybridization of a double-stranded oligonucleotide functionalized on a nanoparticle to a single-stranded target polynucleotide.

In some embodiments, the disclosure contemplates that a polynucleotide attached to a nanoparticle is RNA. The RNA can be either single-stranded or double-stranded, so long as it is able to hybridize to a target polynucleotide.

In some aspects, multiple polynucleotides are functionalized to a nanoparticle. In various aspects, the multiple polynucleotides each have the same sequence, while in other aspects one or more polynucleotides have a different sequence. In further aspects, multiple polynucleotides are arranged in tandem and are separated by a spacer. Spacers are described in more detail herein below.

Polynucleotide Attachment to a Nanoparticle.

Polynucleotides contemplated for use in the methods include those bound to the nanoparticle through any means (e.g., covalent or non-covalent attachment). Regardless of the means by which the polynucleotide is attached to the nanoparticle, attachment in various aspects is effected through a 5′ linkage, a 3′ linkage, some type of internal linkage, or any combination of these attachments. In some embodiments, the polynucleotide is covalently attached to a nanoparticle. In further embodiments, the polynucleotide is non-covalently attached to a nanoparticle. An oligonucleotide of the disclosure comprises, in various embodiments, an associative moiety selected from the group consisting of a tocopherol, a cholesterol moiety, DOPE-butamide-phenylmaleimido, and lyso-phosphoethanolamine-butamide-pneylmaleimido. See also U.S. Patent Application Publication No. 2016/0310425, incorporated by reference herein in its entirety.

Methods of attachment are known to those of ordinary skill in the art and are described in US Publication No. 2009/0209629, which is incorporated by reference herein in its entirety. Methods of attaching RNA to a nanoparticle are generally described in PCT/US2009/65822, which is incorporated by reference herein in its entirety. Methods of associating polynucleotides with a liposomal particle are described in PCT/US2014/068429, which is incorporated by reference herein in its entirety.

Peg Density.

A density of PEG in association with the nanoparticle is shown herein to modulate the cellular uptake of uncleaved PEG structures. In addition to PEG, the disclosure contemplates the use of other molecules that prevent opsonization. For example and without limitation, the disclosure contemplates the use of polysaccharides (e.g., dextran) in place of or in addition to PEG. In general, the disclosure contemplates that high densities of shorter PEG polymers reduces cellular uptake in a fashion similar to longer PEG polymers. In various embodiments, the PEG density is from about 1 to about 80 pmol/cm². Methods are also provided wherein the PEG density is at least 1.5 pmol/cm², 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm2, at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², or more.

Oligonucleotide Surface Density.

An oligonucleotide surface density adequate to make the nanoparticles stable and the conditions necessary to obtain it for a desired combination of nanoparticles and polynucleotides can be determined empirically. Generally, a surface density of at least about 2 pmoles/cm² will be adequate to provide stable nanoparticle-oligonucleotide compositions. In some aspects, the surface density is at least 15 pmoles/cm². Methods are also provided wherein the oligonucleotide is bound to or associated with the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm2, at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

Alternatively, the density of oligonucleotide on the surface of the SNA is measured by the number of oligonucleotides on the surface of a SNA. With respect to the surface density of oligonucleotides on the surface of a SNA of the disclosure, it is contemplated that a SNA as described herein comprises from about 1 to about 300 oligonucleotides on its surface. In various embodiments, a SNA comprises from about 10 to about 300, or from about 10 to about 250, or from about 10 to about 200, or from about 10 to about 150, or from about 10 to about 100, or from 10 to about 90, or from about 10 to about 80, or from about 10 to about 70, or from about 10 to about 60, or from about 10 to about 50, or from about 10 to about 40, or from about 10 to about 30, or from about 10 to about 20 oligonucleotides on its surface. In further embodiments, a SNA comprises at least about 5, 10, 20, 30, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 oligonucleotides on its surface.

PEG Molecular Weight/Length.

In various embodiments, it is contemplated that PEG from about 200 Daltons to about 10,000 Daltons is useful in the methods and compositions (e.g., compositions comprising a SNA as disclosed herein) of the disclosure. In further embodiments, PEG that is at least 200, at least 250, at least 300, at least 350, at least 400, at least 500, at least 600, at least 700, at least 800, at least 900, at least 1000, at least 1500, at least 2000, at least 2500, at least 3000, at least 3500, at least 4000, at least 4500, at least 5000, at least 5500, at least 6000, at least 6500, at least 7000, at least 7500, at least 8000, at least 8500, at least 9000, at least 9500, at least 10000 daltons, or more is contemplated. In still further embodiments, PEG that is from about 200 to about 500 daltons, or from about 200 to about 1000 daltons, or from about 1000 daltons to about 5000 daltons, or from about 1000 to about 7000 daltons, or from about 5000 to about 10000 daltons is contemplated.

Cleavable Peptide Linker Sequences.

As disclosed herein, in any of the aspects of the disclosure a cleavable linker is used to attach PEG to the SNA. In some embodiments, the cleavable linker comprises a peptide sequence that is recognized and cleaved by a specific enzyme. The use of a cleavable peptide linker sequence allows for the generation of SNAs that possess the properties of increased in vivo circulation time while maintaining high cellular uptake. In addition, the programmability of PEG cleavage (e.g., via modulating peptide sequence, PEG density, and/or PEG length) can be used to create SNAs that activate at different times within the TME to maintain therapeutic dosing over extended time periods. Cleavable peptide linker sequences contemplated by the disclosure include PLGLAG (SEQ ID NO: 1), PQGIAGW (SEQ ID NO: 2), KPLGLAR (SEQ ID NO: 3), PLGMYSR (SEQ ID NO: 4), and PLGMSR (SEQ ID NO: 5). It is contemplated herein that peptide sequences can be chosen to either control the rate of cleavage or to modulate the specificity to different MMPs, e.g. MMP9, MMP2, MMP7, or a combinations of these enzymes.

Spacers.

In certain aspects, functionalized nanoparticles are contemplated which include those wherein an oligonucleotide or a peptide-PEG moiety is attached to the nanoparticle through a “spacer.” “Spacer” as used herein is a moiety that serves to increase distance between the nanoparticle and the oligonucleotide or the peptide-PEG moiety. In some aspects, the spacer when present is an organic moiety. In further aspects, the spacer is a polymer, including but not limited to a water-soluble polymer, a nucleic acid, a polypeptide, an oligosaccharide, a carbohydrate, a lipid, an ethylglycol, or combinations thereof. In some embodiments, the spacer is PEG.

Thus, in certain aspects, the oligonucleotide has a spacer through which it is covalently bound to the nanoparticles. As a result of the binding of the spacer to the nanoparticles, the oligonucleotide or the peptide-PEG moiety is spaced away from the surface of the nanoparticles. In various embodiments, the length of the spacer is or is equivalent to at least about 5 nucleotides, 5-10 nucleotides, 10 nucleotides, 10-30 nucleotides, or even greater than 30 nucleotides. The spacer may have any sequence which does not interfere with the ability of the polynucleotides to become bound to the nanoparticles or to a target polynucleotide. In certain aspects, the bases of a polynucleotide spacer are all adenylic acids, all thymidylic acids, all cytidylic acids, all guanylic acids, all uridylic acids, or all some other modified base.

Uses of SNAs in Gene Regulation Therapy.

It is contemplated that in some embodiments, a SNA of the disclosure possesses the ability to regulate gene expression. The nucleic acids utilized in the synthesis of the SNA include specific sequences that can be used to regulate the expression of specific proteins by cells to modulate cell behavior (e.g., slow proliferation, induce cell death). For example and without limitation, it is contemplated that SNAs are produced to include specific sequences that are used to regulate the expression of Bcl2L12 (an oncoprotein overexpressed in glioblastoma relative to normal brain), isocitrate dehydrogenase (NADP(+)) 1 (IDH1), and/or human epidermal growth factor receptor 2 (Her2). Thus, in some embodiments, a SNA of the disclosure comprises an oligonucleotide having gene regulatory activity (e.g., inhibition of target gene expression or target cell recognition). Accordingly, in some embodiments the disclosure provides methods for inhibiting gene product expression, and such methods include those wherein expression of a target gene product is inhibited by about or at least about 5%, about or at least about 10%, about or at least about 15%, about or at least about 20%, about or at least about 25%, about or at least about 30%, about or at least about 35%, about or at least about 40%, about or at least about 45%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 96%, about or at least about 97%, about or at least about 98%, about or at least about 99%, or 100% compared to gene product expression in the absence of a SNA. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of SNA and a specific oligonucleotide.

In various aspects, the methods include use of an oligonucleotide which is 100% complementary to the target polynucleotide, i.e., a perfect match, while in other aspects, the oligonucleotide is about or at least (meaning greater than or equal to) about 95% complementary to the polynucleotide over the length of the oligonucleotide, about or at least about 90%, about or at least about 85%, about or at least about 80%, about or at least about 75%, about or at least about 70%, about or at least about 65%, about or at least about 60%, about or at least about 55%, about or at least about 50%, about or at least about 45%, about or at least about 40%, about or at least about 35%, about or at least about 30%, about or at least about 25%, about or at least about 20% complementary to the polynucleotide over the length of the oligonucleotide to the extent that the oligonucleotide is able to achieve the desired degree of inhibition of a target gene product. Moreover, an oligonucleotide may hybridize over one or more segments such that intervening or adjacent segments are not involved in the hybridization event (e.g., a loop structure or hairpin structure). The percent complementarity is determined over the length of the oligonucleotide. For example, given an inhibitory oligonucleotide in which 18 of 20 nucleotides of the inhibitory oligonucleotide are complementary to a 20 nucleotide region in a target polynucleotide of 100 nucleotides total length, the oligonucleotide would be 90 percent complementary. In this example, the remaining noncomplementary nucleotides may be clustered or interspersed with complementary nucleobases and need not be contiguous to each other or to complementary nucleotides. Percent complementarity of an inhibitory oligonucleotide with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Accordingly, methods of utilizing a SNA of the disclosure in gene regulation therapy are provided. This method comprises the step of hybridizing a polynucleotide encoding the gene with one or more oligonucleotides complementary to all or a portion of the polynucleotide, wherein hybridizing between the polynucleotide and the oligonucleotide occurs over a length of the polynucleotide with a degree of complementarity sufficient to inhibit expression of the gene product. The inhibition of gene expression may occur in vivo or in vitro.

The oligonucleotide utilized in the methods of the disclosure is either RNA or DNA. The RNA can be an inhibitory RNA (RNAi) that performs a regulatory function, and in various embodiments is selected from the group consisting of a small inhibitory RNA (siRNA), an RNA that forms a triplex with double stranded DNA, and a ribozyme. Alternatively, the RNA is microRNA that performs a regulatory function. The DNA is, in some embodiments, an antisense-DNA.

Agents.

In some aspects, the disclosure contemplates an oligonucleotide-functionalized MOF nanoparticle further comprising an agent. In various embodiments, the agent is a peptide, a protein, an antibody, a small molecule, or a combination thereof. In any of the embodiments of the disclosure, the agent is encapsulated in the nanoparticle. Methods of encapsulating an agent in a nanoparticle are generally known in the art [Li, P.; Klet, R. C.; Moon, S. Y.; Wang, T. C.; Deria, P.; Peters, A. W.; Klahr, B. M.; Park, H. J.; Al-Juaid, S. S.; Hupp, J. T.; Farha, O. K. Chem. Commun. 2015, 51, 10925-10928; Kelty, M. L.; Morris, W.; Gallagher, A. T.; Anderson, J. S.; Brown, K. A.; Mirkin, C. A.; Harris, T. D. Chem. Commun. 2016, 52, 7854-7857].

An “agent” as used herein means any compound useful for therapeutic or diagnostic purposes. The term as used herein is understood to include any compound that is administered to a patient for the treatment or diagnosis of a condition.

Protein therapeutic agents include, without limitation peptides, enzymes, structural proteins, receptors and other cellular or circulating proteins as well as fragments and derivatives thereof, the aberrant expression of which gives rise to one or more disorders. Therapeutic agents also include, as one specific embodiment, chemotherapeutic agents. Therapeutic agents also include, in various embodiments, a radioactive material.

In various aspects, protein therapeutic agents include cytokines or hematopoietic factors including without limitation IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, erythropoietin (EPO), thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2a, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein α, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor receptor, TNF, including INFO, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof. Examples of biologic agents include, but are not limited to, immuno-modulating proteins such as cytokines, monoclonal antibodies against tumor antigens, tumor suppressor genes, and cancer vaccines. Examples of interleukins that may be used in conjunction with the compositions and methods of the present invention include, but are not limited to, interleukin 2 (IL-2), and interleukin 4 (IL-4), interleukin 12 (IL-12). Other immuno-modulating agents other than cytokines include, but are not limited to bacillus Calmette-Guerin, levamisole, and octreotide.

In various embodiments, therapeutic agents described in U.S. Pat. No. 7,667,004 (incorporated by reference herein in its entirety) are contemplated for use in the compositions and methods disclosed herein and include, but are not limited to, alkylating agents, antibiotic agents, antimetabolic agents, hormonal agents, plant-derived agents, and biologic agents.

Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (e.g., carboplastin, cisplatin and platinum (IV) (Pt(IV))).

Examples of antibiotic agents include, but are not limited to, anthracyclines (e.g. doxorubicin, daunorubicin, epirubicin, idarubicin and anthracenedione), mitomycin C, bleomycin, dactinomycin, plicatomycin. Additional antibiotic agents are discussed in detail below.

Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, imatinib mesylate (or GLEEVEC®), and gemcitabine.

Examples of hormonal agents include, but are not limited to, synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone.

Examples of plant-derived agents include, but are not limited to, vinca alkaloids (e.g., vincristine, vinblastine, vindesine, vinzolidine and vinorelbine), podophyllotoxins (e.g., etoposide (VP-16) and teniposide (VM-26)), camptothecin compounds (e.g., 20(S) camptothecin, topotecan, rubitecan, and irinotecan), taxanes (e.g., paclitaxel and docetaxel).

Chemotherapeutic agents contemplated for use include, without limitation, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2″-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinum coordination complexes such as cisplatin, Pt(IV) and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

Chemotherapeutics also include, but are not limited to, an anti-PD-1 antibody, alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics (e.g., cisplatin), polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, topoisomerase inhibitors, ubiquitin ligase inhibitors, and the like, as well as combinations of one or more of these agents. Additional chemotherapeutics are disclosed in U.S. Patent Application Publication No. 2018/0072810, incorporated by reference herein in its entirety.

In some embodiments, agents include small molecules. The term “small molecule,” as used herein, refers to a chemical compound, for instance a peptidometic that may optionally be derivatized, or any other low molecular weight organic compound, either natural or synthetic. Such small molecules may be a therapeutically deliverable substance or may be further derivatized to facilitate delivery.

By “low molecular weight” is meant compounds having a molecular weight of less than 1000 Daltons, typically between 300 and 700 Daltons. Low molecular weight compounds, in various aspects, are about 100, about 150, about 200, about 250, about 300, about 350, about 400, about 450, about 500, about 550, about 600, about 650, about 700, about 750, about 800, about 850, about 900, or about 1000 Daltons.

Compositions.

The disclosure includes compositions that comprise a pharmaceutically acceptable carrier and a spherical nucleic acid (SNA) of the disclosure, wherein the SNA comprises an oligonucleotide functionalized thereto, the oligonucleotide comprising polyethylene glycol (PEG) and/or a peptide, configured as follows:

nanoparticle------oligonucleotide------peptide------PEG.

In some embodiments, the composition is an antigenic composition. The term “carrier” refers to a vehicle within which the SNA is administered to a mammalian subject. The term carrier encompasses diluents, excipients, adjuvants and combinations thereof. Pharmaceutically acceptable carriers are well known in the art (see, e.g., Remington's Pharmaceutical Sciences by Martin, 1975).

Exemplary “diluents” include sterile liquids such as sterile water, saline solutions, and buffers (e.g., phosphate, tris, borate, succinate, or histidine). Exemplary “excipients” are inert substances include but are not limited to polymers (e.g., polyethylene glycol), carbohydrates (e.g., starch, glucose, lactose, sucrose, or cellulose), and alcohols (e.g., glycerol, sorbitol, or xylitol).

Adjuvants include but are not limited to emulsions, microparticles, immune stimulating complexes (iscoms), LPS, CpG, or MPL.

Each of the references cited herein is incorporated by reference in its entirety, or as relevant in view of the context of the citation.

### EXAMPLES

### Example 1

### Synthesis of the Conjugates

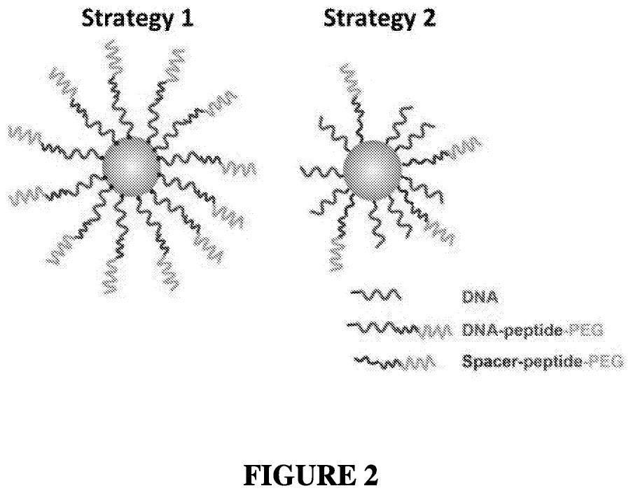

Two approaches to making the cleavable PEG conjugates are provided. The first approach involves the synthesis of an oligonucleotide-peptide-PEG conjugate and its subsequent conjugation to the surface of the nanoparticle via the oligonucleotide. The second approach involves co-functionalizing the particle with oligonucleotides along with PEG-peptide conjugates through either direct attachment of a peptide-PEG to the surface or the use of spacer PEG or amino acids (Spacer) between the nanoparticle surface and the conjugate (FIG. 2). Significantly, in the second strategy, when a spacer is used, the Spacer, which will attach to the surface of the particle, should be shorter in length than the oligonucleotide. In various embodiments, the Spacer is less than about 75%, less than 70%, less than 65%, less than 60%, less than 55%, less than 50%, less than 45%, less than 40%, less than 35%, less than 30%, less than 25%, less than 20%, less than 15%, less than 10%, less than 5%, or less than 1% of the DNA length. This shorter length allows the oligonucleotide to readily interact with cells after the peptide is enzymatically cleaved.

For both strategies, an MMP cleavable substrate containing a specific amino acid motif is synthesized and incorporated into the SNA structure. Numerous sequences have been identified for different MMPs [Nagase, Substrate specificity of MMPs. In Matrix Metalloproteinase Inhibitors in Cancer Therapy, Springer: 2001; pp 39-66]. Herein, the well-established sequence PLGLAG (SEQ ID NO: 1) is utilized, which is cleaved by MMP-2/MMP-9 with cleavage occurring between the G and L [Olson et al., Proceedings of the National Academy of Sciences 2010, 107 (9), 4311-4316; Aguilera et al., Integrative Biology 2009, 1 (5-6), 371-381; Slack et al., Chemical Communications 2017, 53 (6), 1076-1079; Jiang et al., Proc. Natl. Acad. Sci. U.S.A 2004, 101 (51), 17867-17872]. In order to allow the synthesis of larger conjugates, two orthogonal functional groups were incorporated on either end of the peptide. The first functional group for conjugation is an amine in a lysine (K). The second functional group is incorporated via a modified lysine containing an azide (K{N₃}). For peptide-PEG conjugates that are directly functionalized to gold nanoparticle surface a cysteine (C) was used in place of the K{N₃}. Importantly, any compatible and orthogonal functional groups can be incorporated into the peptides for functionalization to PEG or oligonucleotides, including alkenes, amines, carboxylic acids, thiols, alkynes, and azides.

For initial cleavage and cell uptake studies, a fluorophore-quencher pair consisting of either methoxycoumarin (Mca) on the N-terminus and a 2,4-dinitrophenol modified amino acid (Dap{Dnp}) or N-terminal 2-Aminobenzoyl (Abz) and a nitro-tyrosine (Y{NO₂}) amino acid were incorporated into the structure. Table 1 contains detailed sequences. Control sequences consisting of amino acids in the cleavage domain (PLGLAG (SEQ ID NO: 1)) substituted for their D-enantiomers were used to ensure that cleavage is specific; MMPs cannot cleave these enantiomers due to their stereoselectivity [Jiang et al., Proc. Natl. Acad. Sci. U.S.A 2004, 101 (51), 17867-17872].

TABLE 1
Cleavable peptide substrates.

| | | | SEQ ID |
|---|---|---|---|
| Peptide | Use | Sequence | NO: |
| FR1 | Oligo/PEG(1)-Peptide- | Mca-KGPLGLAG(Dap{Dnp})(K{N₃})-CONH₂ | 6 |
| | PEG | | |
| FR2 | Oligo/PEG(1)-Peptide- | Mca-KGPLGLA(Dap{Dnp})G(K{N₃})-CONH₂ | 7 |
| | PEG | | |
| FR3 | Peptide-PEG Directly on | Abz-KPLGLAG(Y{NO₂})C-COOH | 8 |
| | Au NP | | |
| P1 | Oligo/PEG(1)-Peptide- | CH₃-KGPLGLAGG(K{N₃})-CONH₂ | 9 |
| | PEG | | |
| P2 | Oligo/PEG(1)-Peptide- | CH₃-KGPLGLAGGC-CONH₂ | 10 |
| | PEG | | |

### Peptide-PEG Synthesis

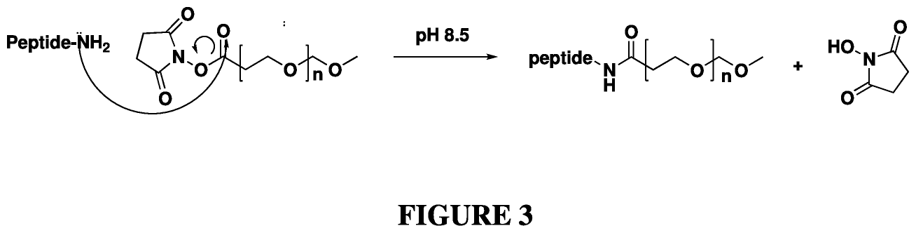

Conjugation of a 2 kD PEG to the peptides was performed by mixing an N-Hydroxysuccinimide (NHS) ester modified PEG with the peptide. To perform this reaction, the peptide and PEG were suspended in a 75% (v/v) acetonitrile (MeCN) solution in water with 10 mM of HEPES buffered to pH 8.5 (FIG. 3). The reaction was allowed to proceed overnight on a shaker at room temperature. Following overnight shaking, the solution was lyophilized. The peptide-PEG conjugates were purified using high performance liquid chromatography (HPLC) on a Varian ProStar 210 (Agilent Technologies Inc., Palo Alto, Calif., USA) equipped with a reverse-phase semi-preparative Varian column (Agilent Technologies, 250 mm×10 mm, Microsorb 300 Å/10 μm/C4, gradient=0.1% v/v trifluoractetic acid (TFA) (aq) to 70% pure 0.1% TFA in MeCN over 40 min, flow rate=3 mL/min). The fractions were concentrated on a lyophilizer overnight and the molecular weight of the conjugated was verified with mass spectroscopy (matrix-assisted laser desorption/ionization time-of-flight (MALDI-TOF) mass spectroscopy).

### Synthesis of Oligonucleotides

Oligonucleotides were synthesized on CPG supports using an automated nucleotide system (model: MM12, BioAutomation Inc., Plano, Tex., USA). Whenever a modified (i.e., non-nucleoside-bearing) phosphoramidite was used, the coupling time was either extended to 10 min compared to the usual 90 seconds or done by hand with a coupling time of 4-16 hours. After synthesis, the completed DNA was cleaved off the CPG support through a 17 hour exposure to aqueous ammonium hydroxide (28-30 wt %). Ammonium hydroxide was removed from the cleaved DNA solution by passing a stream of dry nitrogen gas over the contents of the vial until the characteristic ammonia smell disappeared. The remaining solution was passed through a 0.2 μm cellulose acetate membrane filter to remove the solid support and then purified on a Varian ProStar 210 with a reverse-phase semi-preparative Varian column (250 mm×10 mm, Microsorb 300 Å/10 μm/C4 (for dye-modified oligonucleotides and alkyne terminated) or C18 for all other sequences), gradient=95:5 v/v 0.1 M TEAA (aq):MeCN (TEAA (aq)=triethylammonium acetate, aqueous solution), and increasing to 75% (v/v) MeCN in 45 min, flow rate=15 mL/min).

Initial sequences used in synthesis of the conjugates are shown in Table 2. Significantly, the sequence can be changed to include any targeting sequence for gene regulation.

TABLE 2
Oligonucleotide Sequences.

| Strand | Sequence (5′ to 3′) | SEQ ID NO: |
|---|---|---|
| C8 Alkyne-T20 | C8 Alkyne-TTTTTTTTTTTTTTTTTTTT-Sp18-Sp18-SH | 11 |
| Hexynyl-T20 | Hexynyl-TTTTTTTTTTTTTTTTTTTT-Sp18-Sp18-SH | 12 |
| Cy5 Labeled | C8 Alkyne-Cy5-TTTTTTTTTTTTTTTTTTTT-Sp18-Sp18-SH | 13 |
| Cy3.5 labeled | Cy3.5-TTTTTTTTTTTTTTTTTTTT-Sp18-Sp18-SH | 14 |

C8 Alkyne = 5′-Dimethoxytrityl-5-(octa-1,7-diynyl)-2′-deoxyuridine, 3′-[(2-cyanoethyl)-(N,N-diisopropyl)]-phosphoramidite; Hexynyl = 5-Hexyn-1-yl-(2-cyanoethyl)-(N,N-diisopropyl)-phosphoramidite; Cy5 = 1-[3-(4-monomethoxytrityloxy)propyl]-1′-[3-[(2-cyanoethyl)-(N,N-diisopropyl phosphoramidityl]propyl]-3,3,3′,3′-tetramethylindodicarbocyanine chloride; Cy3.5 = 1-[3-(4-monomethoxytrityloxy)propyl]-1′-[3-[(2-cyanoethyl)-(N,N-diisopropyl phosphoramidityl]propyl]-3,3,3′,3′-tetramethylindodicarbocyanine chloride; SH = 1-O-Dimethoxytrity1-3-oxahexyl-disulfide,1′-succinoyl-long chain alkylamino-CPG

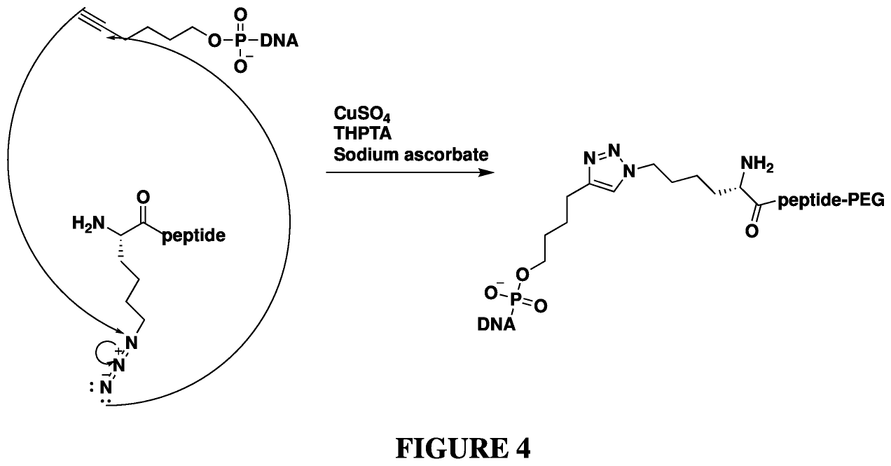

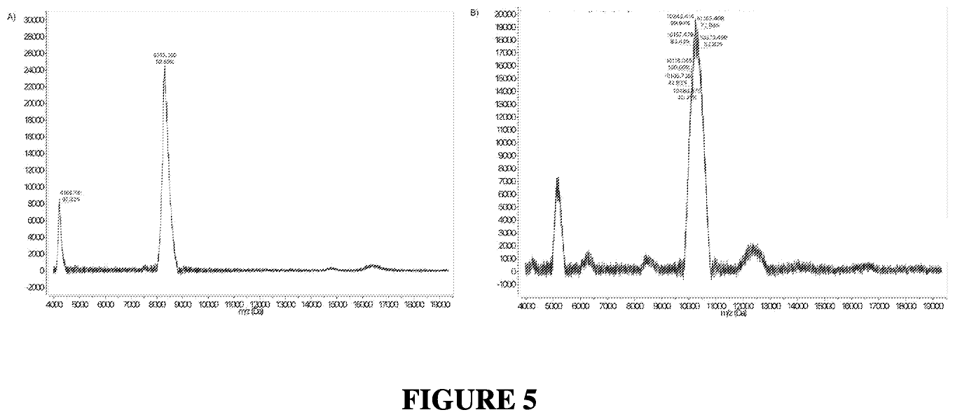

For conjugation of the oligonucleotides to peptide-PEG conjugates, copper mediated alkyne-azide cycloaddition click chemistry was utilized [Rostovtsev et al., Angewandte Chemie 2002, 114 (14), 2708-2711]. Briefly, tris(3-hydroxypropyltriazolylmethyl)amine (THPTA) was added (1:1) to the CuSO₄ to stabilize Cu(I) ions. Oligonucleotides containing a 3′ thiol and a 5′ alkyne were then mixed with 5 equivalents (eq) of azide containing peptide-PEG conjugates; the reaction mixture, 25 eq of CuSO₄/THPTA and 40 eq of sodium ascorbate were added sequentially (FIG. 4). The reaction proceeded overnight at room temperature. The DNA-peptide-PEG conjugates were then HPLC purified using the same procedure as the peptide-PEG conjugates. Following purification, the fractions were concentrated using lyophilization and the purified product fraction was identified via MALDI-TOF MS (FIG. 5).

Similarly, PEG(1)-Peptide-PEG(2) conjugates can be constructed using the same procedure by taking the peptide-PEG conjugate and substituting an alkyne modified PEG terminated with a thiol for the alkyne modified oligonucleotides.

### Synthesis of SNAs

Thirteen nanometer (nm) citrate stabilized gold nanoparticles (Au NPs) were synthesized following previously established methods [Cutler et al., Journal of the American Chemical Society 2012, 134 (3), 1376-1391; Mirkin et al., Nature 1996, 382 (6592), 607-609]. The Au NPs were functionalized with oligonucleotides and their respective conjugate either through established salt-aging or freeze-thaw procedures [Cutler et al., Journal of the American Chemical Society 2012, 134 (3), 1376-1391; Mirkin et al., Nature 1996, 382 (6592), 607-609; Liu et al., J. Am. Chem. Soc. 2017, 139 (28), 9471-9474]. The density of the PEG shell can be modulated by incubating the Au NPs with different ratios of thiol terminated DNA and the desired PEG conjugate.

To determine if the attachment of peptide-PEG conjugates to oligonucleotides interfered with oligonucleotide loading, the number of strands per an Au NP was evaluated using an OliGreen assay (ThermoFisher Scientific). The particles were treated with 1 M potassium cyanide (KCN) to dissolve the Au and release the oligonucleotides. The assay was then conducted following procedures outlined by the manufacturer. Upon measuring the loading, the strand density was found to be approximately 30 pmol/cm² and approximately 21 pmol/cm² for L- and D-enantiomer peptides respectively.

For structures with different ratios of oligonucleotide to PEG conjugate, the relative and total loading was determined by using fluorophore labeled oligonucleotides. Cy3.5 labeled oligonucleotides were functionalized to peptide-PEG and unconjugated Cy5 labeled oligonucleotides were used in excess (sequences; Table 2). The relative loading of the two structures was then determined by dissolving the Au NPs with KCN (5 mM) and measuring the fluorescence.

Enzyme Activity Assay

The ability of the enzymes to cleave PEG from nanoparticles was evaluated by treating the structures with either MMP-2 or -9. The samples were pipetted into wells in a 96 well plate format. The concentration of peptide was held between 0.01 and 0.14 μM for each sample. Twenty nanograms (ng) of MMP was added to each well and the samples were heated to 37° C. in a plate reader. The fluorescence was monitored over time (320 nm excitation; 392 nm emission; slit width=17 nm).

### Example 2

### Synthesis of SNAs with Peptide-PEG Conjugates Attached to the AuNP Core

For SNAs featuring peptide(P3)-PEG conjugates attached directly to AuNPs cores, the azide functional groups in the peptide were conjugated to a Dibenzocyclooctyne (DBCO) attached to PEG polymers. PEG polymers with average molecular weights of 2000 Da, 5000 Da, and 10000 Da (BroadPharm, San Diego, Calif., USA) were conjugated to peptides by mixing the two reagents in water overnight with constant shaking at room temperature. The product was purified by a size exclusion chromatography, using a peptide column (GE Healthcare Life Sciences, Chicago, Ill., USA) that was connected to a medium-pressure liquid chromatography system (Bio-Rad, Hercules, Calif., USA). The purified fractions were collected, lyophilized, and verified for molecular weight by MALDI-TOF mass spectrometry.

For fluorescence-based assays measuring the loading density, fluorophores were attached to the peptide(P3)-PEG conjugates. To accomplish this, the N-terminus was labeled by adding the peptide to excess NHS-ester modified BODIPY 650/665-X dye (at approximately 1:2 ratio) in dimethylformamide (DMF) at room temperature overnight. Unconjugated dyes were removed by passing the reaction mixture through a manually-packed Sephadex G-10 column (GE Healthcare Life Sciences, Chicago, Ill., USA). The product fraction was the first band eluted from the column and was collected and reacted with DBCO-modified PEG with average molecular weights of 2000 Da, 5000 Da, and 10000 Da. The reaction, purification, and characterization procedure was performed as disclosed herein.

A backfilling salt-aging method was used to functionalize AuNPs with different peptide conjugates of different PEG molecular weights following methods outlined previously [Chinen et al., Bioconjugate Chemistry 2016, 27, (11), 2715-2721]. Briefly, peptide(P3)-PEG conjugates, consisting of PEGs of different average molecular weights and oligonucleotides (Thiol-T20 or Thiol-1826) were incubated with AuNPs at a ratio of 1:5. During the incubation, NaCl was added in 3 increments every 30 minutes, raising the salt concentration from 0 mM initial to 0.2 M, 0.3 M, and finally 0.5M.

### Quantification of Loading

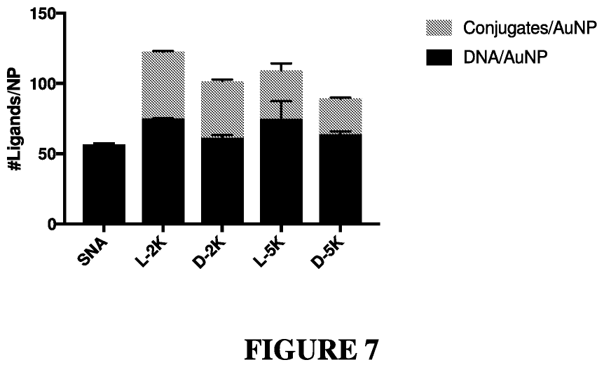

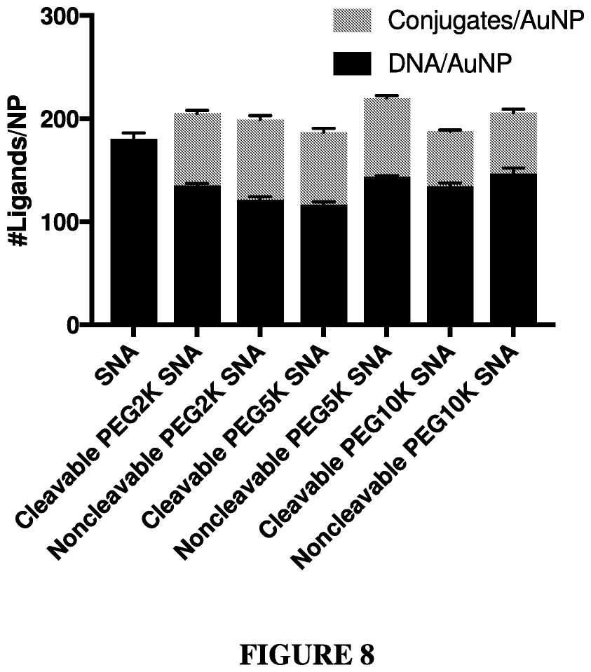

To determine the loading density of PEG and oligonucleotides, fluorophore-labeled oligonucleotides and conjugates were used according to methods disclosed herein in Example 1. A calibration curve was generated by measuring the fluorescence of known concentrations of Cy3.5 labeled oligonucleotides and BODIPY 650/665-X-labeled peptide-PEG conjugates. The loading density of the two ligands on the AuNP was then determined by measuring the Cy3.5 fluorescence (Excitation: 581 nm; Emission: 600 nm, slit width 9 nm) and BODIPY fluorescence (Excitation 650 nm; Emission 670 nm, slit width 9 nm) after digestion of the AuNP core using KCN (0.1 M) (BioTek Instruments, Inc., Winooski, Vt., USA). The number of strands was determined by comparing to a linear curve generated from the standards. The number of strands of oligonucleotides ranged between (80-130 per AuNP) and the number of PEG conjugates was between (20-50 per AuNP), see FIGS. 7 and 8, underscoring the ability to produce structures with similar loading of oligonucleotides and PEGs functionalized to the AuNP core.

### Enzyme Cleavage of Low-Density PEG Functionalized Directly to SNA Core

The first focus was to examine how PEG length affects peptide cleavage kinetics, dictating PEG removal. For initial studies, PEGs of varying molecular weights (2, 5, and 10 kD molecular weight) were attached to the MMP-responsive peptide (P3). The peptide-PEG conjugates were purified using size exclusion chromatography and their conjugation was verified for molecular weight with matrix assisted laser desorption/ionization-time of flight mass spectrometry. SNAs were formed by functionalizing 13-nm Au nanoparticles (AuNPs) with oligonucleotides and cleavable PEG using established salt-aging methods. As controls for the cleavable structures, conventional SNAs containing only oligonucleotides were synthesized, as well as SNAs with non-cleavable PEG shells (‘D’ form of the peptide, see Table 1).

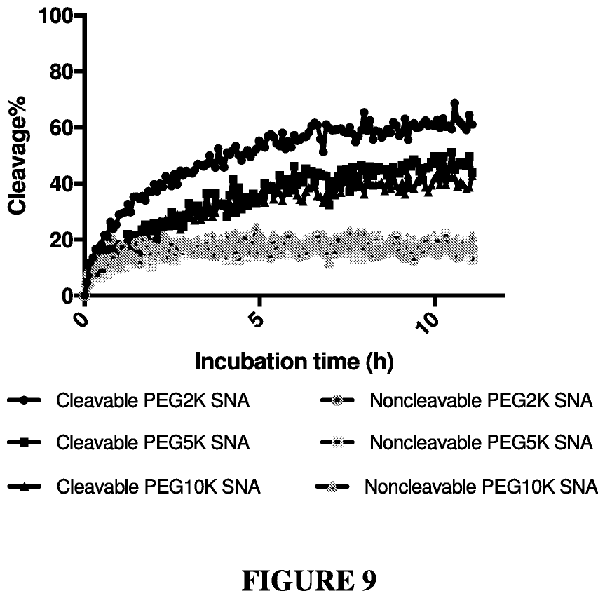

The cleavage of these particles was assessed by attaching a fluorophore to the N-terminus of the linker peptide. With this design, the fluorophore will be quenched when the PEG shell is intact, but will fluoresce when the peptide is cleaved from the particle surface. The studies showed that the rate of PEG removal can be modulated by modifying the length of the polymer chain as seen by the decrease in cleavage rates for 5 kD and 10 kD PEG compared to 2 kD PEG (FIG. 9). This result is consistent with the idea that longer PEG hinders access of the MMPs to the cleavable linker, thereby reducing the rate of PEG shell removal. The ability to modulate the PEG removal kinetics provides a valuable handle for tuning structures for therapeutic purposes. For instance, the programmability of PEG cleavage can be used to create SNAs that activate at different times within the TME to maintain therapeutic dosing over extended time periods.

### Cell Uptake Study

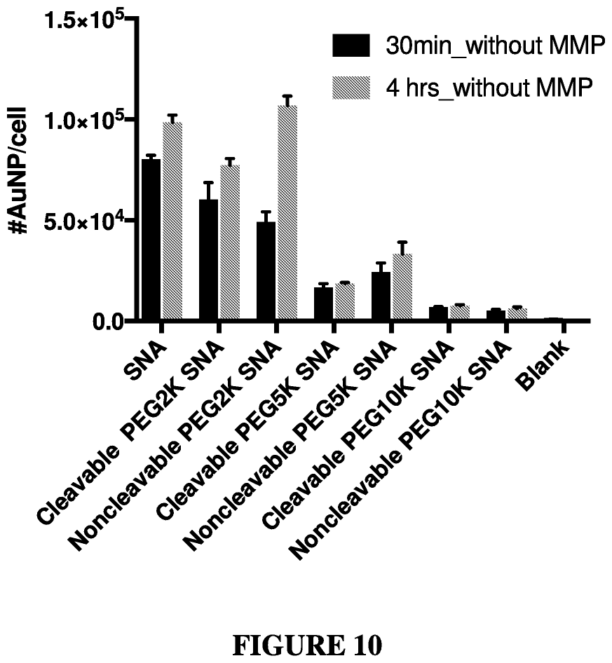

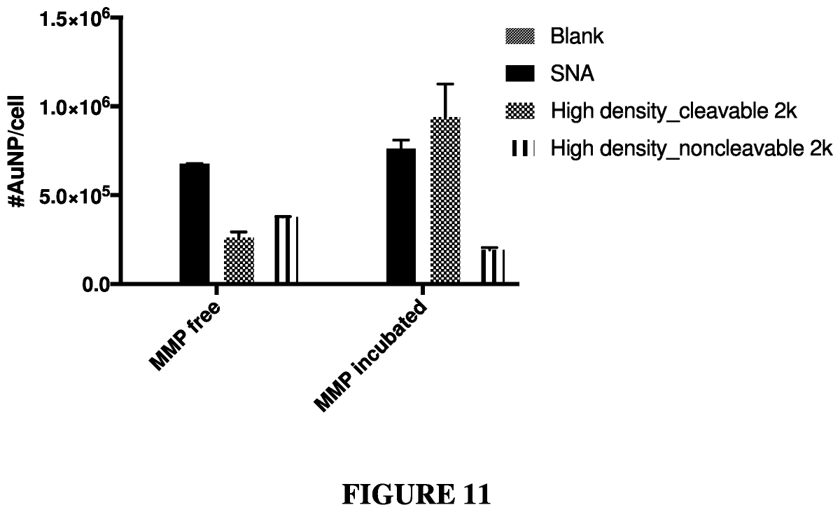

For uptake studies, 4×10⁴ U87 cells were seeded in each well of a 96-well plate. The following day, cells were treated with SNAs functionalized with DNA only, DNA with cleavable conjugates, and DNA with non-cleavable conjugates at different PEG densities or molecular weight (all conjugates consisting of (P3) peptide attached directly to the AuNP core). The concentration of AuNP per well was either 1 nM (FIG. 10) or 3 nM (FIG. 11).

After incubating the SNAs with cells for 30 minutes and 4 hours, the cells were fixed in 4% formaldehyde and a fraction of the cells were stained with a Hoechst stain for cell counting on a flow cytometer. The remaining cells were first digested in concentrated hydrochloric acid and nitric acid, and then Au content was determined by inductively coupled plasma mass spectrometry (ICP-MS). Cell uptake was reported as the number of AuNPs per cell.

To assess whether the PEG layer reduced uptake, cells were treated with PEGylated SNAs consisting of peptide-PEG2K, -PEG5K, and -PEG10K conjugates that were directly attached to Au NPs at lower PEG densities (approximately 50 PEG/AuNP and approximately 125 oligonucleotides/AuNP; FIG. 8). At 30 minutes, the AuNP concentration was reduced for all SNAs coated with PEG, indicating that they had reduced cellular interactions. However, after 4 hours of incubation, conventional SNAs and PEG2K-coated SNAs had similar uptake, which is likely a result of the low steric hindrance of short PEGs at reduced densities. However, PEG5K and PEG10K continued to significantly reduce uptake by cells after 4 hours.

To determine whether high densities of PEG2K could also inhibit cell uptake, cells were treated with high-density peptide-PEG2K SNAs (both cleavable and non-cleavable; approximately 90 PEG/AuNP, approximately 40 oligonucleotides/AuNP). Reduced uptake of the PEG2K coated SNAs was observed after 4 hours of incubation with cells (FIG. 11). This result showed that high densities of shorter PEG polymers can reduce uptake in a fashion similar to longer PEG polymers.

To assess whether enzymes in the TME can restore cell uptake, both the high-density PEGylated and conventional SNAs were treated with MMPs prior to incubation. Importantly, cellular uptake was restored for SNAs formulated with cleavable PEG linkers to levels that mirrored conventional SNAs without a PEG layer after MMP pre-treatment. Additionally, PEGylated SNAs with non-cleavable linkers exhibited no increase in uptake following MMP treatment. Together, these results showed that cleavage of the PEG layer restored the rapid uptake of the SNAs, indicating successful activation of the constructs by proteins excreted by tumor cells. Together, these cell uptake results showed that PEG density and length can be utilized to modulate the cellular uptake of uncleaved PEG structures, and cleavage of the PEG shell can restore cell uptake to levels similar to that of conventional SNAs.

### Biodistribution and Blood Circulation Time Study

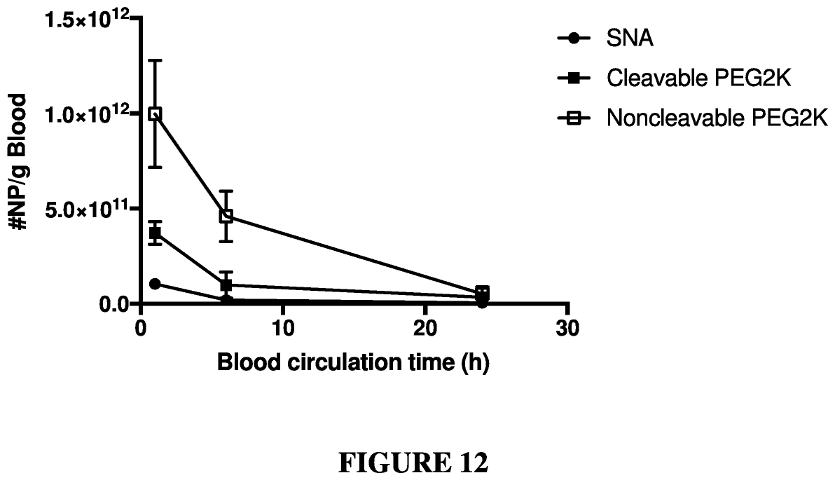

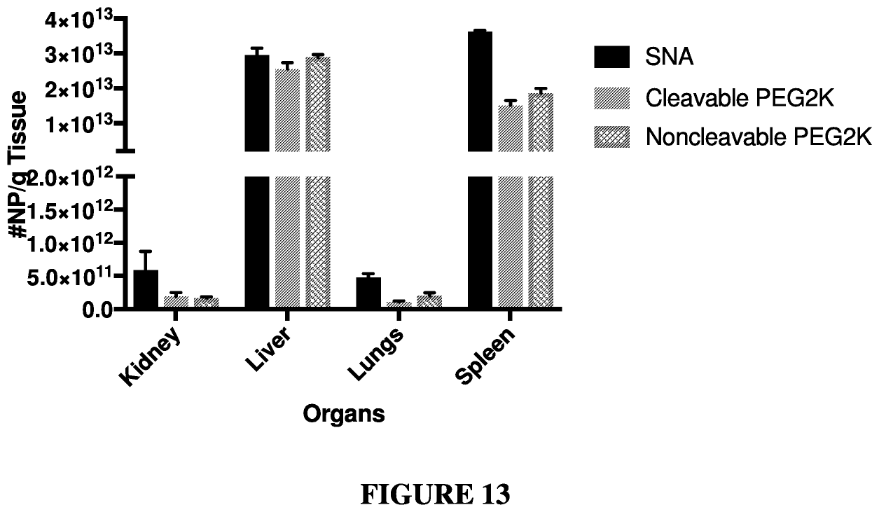

To assess how PEGylated SNAs alter biodistribution, biodistribution studies were performed with MMP-sensitive SNAs in mouse models (500 nM by AuNP, 200 μL per injection). SNAs formulated with cleavable and non-cleavable PEG shells were compared to conventional SNAs without a PEG layer. Mouse models with orthotopic glioblastoma tumors, U87 cells, were treated with each construct. Blood was collected at different time points to assess whether PEGylation with cleavable linkages leads to enhanced blood circulation (FIG. 12). Increased blood circulation times was found for all PEGylated structures compared to conventional SNAs, although after 24 hours nearly all nanomaterials were cleared from the bloodstream regardless of formulation. Following 24 hours of treatment, animals were sacrificed and the organs were harvested to measure the concentration of AuNPs in the tissue via ICP-MS. Decreased accumulation was noted in the spleen, lungs, and heart for PEGylated SNAs (FIG. 13). Together, these results highlighted the ability of PEGylated SNAs to reduce off-target accumulation while increasing blood circulation time.

What is claimed is:

1. A nanoparticle having an oligonucleotide functionalized thereto, the oligonucleotide comprising polyethylene glycol (PEG) and/or a peptide, configured as follows:
nanoparticle------oligonucleotide------peptide------PEG.

2. The nanoparticle of claim 1, further comprising a conjugate functionalized to the nanoparticle, wherein the conjugate comprises a spacer, a peptide and PEG, configured as follows:
nanoparticle------spacer------peptide------PEG.

3. The nanoparticle of claim 2, wherein the spacer comprises PEG or an amino acid.

4. The nanoparticle of claim 3, wherein the spacer is shorter in length than the oligonucleotide.

5. The nanoparticle of any one of claims 1-4, wherein the peptide is enzyme-sensitive.

6. The nanoparticle of claim 5, wherein the enzyme is present in a tumor microenvironment (TME).

7. The nanoparticle of claim 5 or claim 6, wherein the enzyme is a matrix metallo-proteinase (MMP).

8. The nanoparticle of claim 7, wherein the MMP is MMP-2 and/or MMP-9.

9. The nanoparticle of claim 8, wherein the peptide sequence is PLGLAG (SEQ ID NO: 1), PQGIAGW (SEQ ID NO: 2), KPLGLAR (SEQ ID NO: 3), PLGMYSR (SEQ ID NO: 4), or PLGMSR (SEQ ID NO: 5).

10. The nanoparticle of any one of claims 1-9, wherein the nanoparticle is organic or inorganic.

11. The nanoparticle of claim 10, wherein the nanoparticle is metallic.

12. The nanoparticle of claim 11, wherein the nanoparticle comprises gold, silver, platinum, aluminum, palladium, copper, cobalt, indium, or nickel.

13. The nanoparticle of claim 10, wherein the nanoparticle is a liposome.

14. The nanoparticle of any one of claims 1-13, further comprising an agent.

15. A composition comprising the nanoparticle of any one of claims 1-13 and a pharmaceutically acceptable carrier.

16. The composition of claim 15, further comprising an agent.

17. A method of modulating gene expression comprising administering to a cell the nanoparticle of any one of claims 1-14.
